(19) United States
(12) Patent Application Publication
(10) Pub. No.: US 2012/0107900 A1
(43) Pub. Date: May 3, 2012

(54) Electrospun Polymer Fibers Comprising Particles of Bacteria-Containing Hydrogels

(75) Inventors: Andreas Greiner, Amoneburg (DE); Seema Agarwal, Marburg (DE); Marco Gensheimer, Marburg (DE)

(73) Assignee: Philipps-Universitat Marburg, Marburg (DE)

(21) Appl. No.: 13/139,990

(22) PCT Filed: Dec. 18, 2009
(86) PCT No.: PCT/DE2009/001763

§ 371 Date: Dec. 13, 2011

(30) Foreign Application Priority Data
Dec. 19, 2008 (DE) 10 2008 063 821.8

(51) Int. Cl. C12N 11/12; B29C 47/00; C12N 11/08

(52) U.S. Cl. 435/179; 435/182; 264/465

(57) ABSTRACT

The present invention provides electrospun polymer fibers comprising bacteria-containing hydrogel particles. Bacteria-containing hydrogel particles are produced by crosslinking water-soluble polymers to form hydrogels and mixing them with a bacteria suspension. The crosslinking is suitable to be carried out either chemically before the addition of the bacteria suspension or physically before or after this addition. Subsequently, these hydrogel particles are electrospun together with an electrospinnable polymer solution to form fibers or fibre non-wovens.

The bacteria which are located in these hydrogel particles or in the electrospun polymer fibers comprising these particles, respectively, are capable of surviving for a long period (several months) without the supply of water or cell-culture media and are simultaneously protected against the effect of solvents, for example alcohols, acetone, chlorinated hydrocarbons, ethers and toluene, which would otherwise kill said bacteria. The bacteria are suitable to be released again at any time through contact with water and to be replicated under normal culture conditions.

The electrospun polymer fibers according to the present invention comprising bacteria-containing hydrogels are suitable for use in the dry storage of useful bacteria or for killing harmful bacteria. Textiles and membranes, for example, are suitable to be equipped with it. However, they are also suitable for applications in wastewater treatment, environmental protection (water pollution control), the agricultural and food sector, pharmacy, fermentation, and the building industry.

The present invention provides electrospun polymer fibers comprising particles of bacteria-containing hydrogels. The bacteria are packaged into crosslinked hydrogels and are then spun with an electrospinnable polymer to form fibers or fiber nonwovens. In this device according to the present invention, the bacteria are capable of surviving for a long period of time without any supply of water or cell culture media, and are protected against the effect of solvents which would otherwise kill said bacteria. The bacteria can be released again through contact with water or cell culture medium.

### DESCRIPTION OF AND INTRODUCTION TO THE GENERAL FIELD OF THE INVENTION

The present invention relates to the fields of macromolecular chemistry, polymer chemistry, microbiology and material sciences.

### STATE OF THE ART

Useful bacteria, which carry out desired metabolic processes or kill pathogenic germs, are used in various ways, for example in wastewater treatment, water pollution control, the building industry, the agricultural and food sector, pharmacy, fermentation processes and the textile and cosmetics industry.

Until now, the storage of bacteria has required considerable financial outlay and technical effort, for example for the procurement and maintenance of cell culture cabinets and culture vessels, freeze-drying, the freezing and storage of bacteria at very low temperatures and the regular supply of nutrients to the bacteria.

There are also applications in which bacteria must be protected inter alia against the effect of some solvents which are fatal to said bacteria.

Numerous efforts are thus being made to “package” bacteria, so that they are protected against the effect of harmful substances and/or are capable of surviving even in the absence of water or a cell culture medium.

The packaging of bacteria in hydrogel particles made from polyvinyl alcohol (PVA) is described in M Okazaki, T Hamada, H Fujii, A Mizobe and S Matsuzawa, “Development of Poly(vinyl alcohol) Hydrogel for Waste Water Cleaning. I. Study of Poly(vinyl alcohol) Gel as a Carrier for Immobilizing Microorganisms.”, J Appl Polym Sci 1995, 58, 2235-2241 and in M Okazaki, T Hamada, H Fujii, O Kusudo, A Mizobe and S Matsuzawa: “Development of Poly(vinyl alcohol) Hydrogel for Waste Water Cleaning. II. Treatment of N,N-Dimethylformamide in Waste Water with Poly(vinyl alcohol) Gel with Immobilized Microorganisms.” J Appl Polym Sci 1995, 58, 2243-2249. Here, relatively large cell aggregates are packaged, and the resulting bacteria-containing particles are permeable to oxygen and aqueous media.

In “Entrapment in LentiKats®” in “Fundamentals of Cell Immobilisation Biotechnology. Series: Focus on Biotechnology, Vol. 8A”, V Nedovic, R Willaert (Eds.) Springer-Verlag, Heidelberg 2004, pages 53-63 P Wittlich, E Capan, M Schlieker, K-D Vorlop and U Jahnz describe the encapsulation of biocatalysts such as bacteria, fungi, yeasts or enzymes in LentiKats®, i.e. in crosslinked PVA particles. The particles contain individual cells or very small cell aggregates, but have to be stored in aqueous solutions or cell culture media so that the biocatalysts do not die.

DE 10 2005 053 011 A1 describes tetraorganosilicon particles as vesicles for the packaging of active substances. Tetraorganosilicon compounds according to the present invention may be starting materials for the production of hydrogels, and the active substances may optionally be bacteria, bacteria conjugates or bacteria preparations. However, there is no indication of bacteria-containing hydrogel vesicles in which the bacteria are capable of surviving without the supply of air and/or aqueous media.

The production of polymer fibers with diameters in the micrometer and nanometer range is described for example in DE 100 23 456 A1. The production of oriented mesotube and nanotube nonwovens is disclosed in DE 100 53 263 A1.

WO 2008/049250 A1 describes antibacterial electrospun polymer fibers with polyethyleneimine nanoparticles for textile applications. It is disclosed therein how mixtures of particles (here: of polyethyleneimine) and electrospinnable polymers can be jointly spun to form fibers. However, these are antibacterial particles which do not in turn encapsulate any further substances or microorganisms. Moreover, these particles are considerably smaller than the bacteria-containing particles (nm vs. μm).

The present invention overcomes the disadvantages of the state of the art and for the first time provides bacteria-containing hydrogel particles which can be spun jointly with electrospinnable polymers to form fibers and fiber nonwovens. The bacteria in the polymer fibers according to the present invention comprising bacteria-containing hydrogel particles survive for a long period of time without any supply of water or cell culture media and are protected in this device against the effect of solvents which would otherwise kill said bacteria.

### AIM

The aim of the invention is to provide a device in which bacteria survive in the anhydrous state and are protected against the effect of organic solvents, and a method for producing this device.

### ACHIEVEMENT OF THIS AIM

The aim of providing a device in which bacteria survive in the anhydrous state and are protected against the effect of organic solvents is achieved according to the present invention through electrospun polymer fibers comprising particles of bacteria-containing hydrogels.

Surprisingly, it has been found that bacteria survive for a long period of time in the anhydrous state, and are also protected against the effect of organic solvents which would otherwise kill the bacteria if said bacteria are firstly packaged in hydrogel particles and the bacteria-containing hydrogel particles are then incorporated in electrospun fibers.

The device according to the present invention, in which bacteria survive in the anhydrous state and are protected against the effect of organic solvents, and also the method for producing this device are explained hereinafter, wherein the invention comprises all the embodiments presented below individually and in combination with one another.

A hydrogel is a water-containing yet water-insoluble polymer, the molecules of which are chemically or physically linked to form a three-dimensional network. Due to the crosslinking, the ability to swell upon contact with water is provided; no solubility in water is provided. By way of example, but not exhaustively, the hydrogels comprise the following polymers, in each case in crosslinked form: polyvinyl alcohol, polyethylene oxide, polyethyleneimine, polyvinylpyrrolidone, polyacrylic acid, methyl cellulose, hydroxypropyl cellulose, polyacrylamide or partially saponified cellulose acetate. Alternatively, the hydrogel may be starch, wherein starch is branched and not crosslinked.

It is known to persons skilled in the art how water-soluble polymers can be chemically crosslinked. This includes for example irradiation with electron beams or gamma rays and crosslinking agents. These crosslinking agents comprise for example monoaldehydes, dialdehydes, sodium hypochlorite, diisocyanates, dicarboxylic acid halides and chlorinated epoxides.

If hydrogels made from chemically crosslinked polymers are used in the frame of the present invention, the crosslinking agents are preferably selected from monoaldehydes such as acetaldehyde, formaldehyde and dialdehydes such as glutaraldehyde.

If the hydrogel used is chemically crosslinked polyvinyl alcohol, the crosslinking agent used is preferably glutaraldehyde.

It is known to persons skilled in the art which chemical crosslinking agents are particularly suitable for which polymers. Persons skilled in the art are able to apply this knowledge without leaving the scope of protection of the patent claims.

In a preferred embodiment, the hydrogels comprise crosslinked polyvinyl alcohol (PVA). With particular preference, they comprise physically crosslinked PVA.

The physical crosslinking of PVA to form hydrogels takes place for example by repeatedly freezing and thawing a solution of this polymer. Crystallites hereby form in the polymer solution, which act as crosslinking points. Alternatively, the physical crosslinking can also be carried out by dehydrating and then annealing the polymer, since crystallites are also formed as crosslinking points in the process.

Hereinafter, phyla (strains), classes and orders of the bacteria are nominated which, according to the present invention, are suitable to be incorporated into hydrogel particles:
Phylum: Aquificae
   Class: Aquificae
      Order: Aquificales
Phylum: Thermotogae
   Class: Thermotogae
      Order: Thermotogales
Phylum: Thermodesulfobacteria
   Class: Thermodesulfobacteria
      Order: Thermodesulfobacteriales
Phylum: Deionococcus-Thermus
   Class: Deinococci
      Orders: Deionococcales, Thermales
Phylum: Chloroflexi
   Class: Chloroflexi
      Orders: Chloroflexales, Herpetosiphonales
   Class: Anaerolineae
      Order: Anaerolineales
Phylum: Thermomicrobia
   Class: Thermomicrobia
      Order: Thermomicrobiales
Phylum: Nitrospira
   Class: Nitrospira
      Order: Nitrospirales
Phylum: Deferribacteres
   Class: Deferribacteres
      Order: Deferribacterales
Phylum: Cyanobacteria
   Class: Cyanobacteria
      Orders: Subsection I (formerly Chroococcales), Subsection II (Pleurocapsales), Subsection III (Oscillatoriales), Subsection IV (Nostocales), Subsection V (Stigonematales)
Phylum: Chlorobi
   Class: Chlorobia
      Order: Chlorobiales
Phylum: Proteobacteria
   Class: Alphaproteobacteria
      Orders: Rhodospirillales, Rickettsiales, Rhodobacterales, Sphingomonadales, Caulobacterales, Rhizobiales, Parvularculales
   Class: Betaproteobacteria
      Orders: Burkholderiales, Hydrogenophilales, Methylophilales, Neisseriales, Nitrosomonadales, Rhodocyclales, Procabacteriales
   Class: Gammaproteobacteria
      Orders: Chromatiales, Acidithiobacillales, Xanthomonadales, Cardiobacteriales, Thiotrichales, Legionellales, Methylococcales, Oceanospirillales, Pseudomonadales, Alteromonadales, Vibrionales, Aeromonadales, Enterobacteriales, Pasteurellales
   Class: Deltaproteobacteria
      Orders: Desulfurellales, Desulfovibrionales, Desulfobacterales, Desulfarcales, Desulfuromonales, Synthrophobacterales, Bdellovibrionales, Myxococcales (suborders: Cystobacterieae, Sorangineae, Nannocystineae)
   Class: Epsilonproteobacteria
      Order: Campylobacterales
Phylum: Firmicutes
   Class: Clostridia
      Orders: Clostridiales, Thermoanaerobacteriales, Haolanaerobiales
   Class: Mollicutes
      Orders: Mycoplasmatales, Entomoplasmatales, Acholeplasmatales, Anaeroplasmatales, Incertae sedis
   Class: Bacilli
      Orders: Bacillales, Lactobacillales
Phylum: Actinobacteria
   Class: Actinobacteria
      Orders: Acidimicrobiales, Rubrobacterales, Coriobacteriales, Sphaerobacterales, Actinomycetales (suborders: Micorcoccineae, Corynebacterineae, Actinomycineae, Propionibacterineae, Pseudonocardineae, Streptomycineae, Streptomycineae, Micromonosproineae, Frankineae, Glycomycineae), Bifidobacteriales
Phylum: Planctomycetes
   Class: Planctomycetacia
      Order: Planctomycetales
Phylum: Chlamidiae
   Class: Chlamydiae
      Order: Chlamydiales
Phylum: Spirochaetes
   Class: Spirochaetes
      Order: Spirochaetales
Phylum: Fibrobacteres
   Class: Fibrobacteres
      Order: Fibrobacterales
Phylum: Acidobacteria
   Class: Acidobacteria
      Order: Acidobacteriales
Phylum: Bacteroidetes
   Class: Bacteroidetes
      Order: Bacteroidales
   Class: Flavobacteria
      Order: Flavobacteriales
   Class: Sphingobacteria
      Order: Sphingobacteriales
Phylum: Fusobacteria
   Class: Fusobacteria
      Order: Fusobacteriales
Phylum: Verrucomicrobia
   Class: Verrucomicrobiae
      Order: Verrucomicrobiales
Phylum: Dictyoglomi
   Class: Dictyoglomi
      Order: Dictyoglomales
Phylum: Gemmatimonadetes
   Class: Gemmatimonadetes
      Order: Gemmatimonadales

According to the present invention, the electrospun polymer fiber comprises at least one electrospinnable polymer selected from the group poly-(p-xylylene); polyvinylidene halides, polyesters such as polyethylene terephthalate, polybutylene terephthalate; polyethers; polyolefins such as polyethylene, polypropylene, poly(ethylene/propylene) (EPDM); polycarbonates; polyurethanes; natural polymers, e.g. rubber; polycarboxylic acids; polysulfonic acids; sulfated polysaccharides; polylactides such as PLLA; polyglycosides; polyamides; homopolymers and copolymers of aromatic vinyl compounds such as poly(alkyl)styrenes, e.g. polystyrenes, poly-alpha-methylstyrenes; polyacrylonitriles, polymethacrylonitriles; polyacrylamides; polyimides; polyphenylenes; polysilanes; polysiloxanes; polybenzimidazoles; polybenzothiazoles; polyoxazoles; polysulfides; polyesteramides; polyarylene-vinylenes; polyether ketones; polyurethanes, polysulfones, inorganic-organic hybrid polymers such as ORMOCER® from Fraunhofer Gesellschaft zur Förderung der angewandten Forschung e.V. Munich; silicones; fully aromatic copolyesters; poly(alkyl) acrylates; poly(alkyl) methacrylates; polyhydroxyethyl methacrylates; polyvinyl acetates, polyvinyl butyrates such as PVA; polyisoprene; synthetic rubbers such as chlorobutadiene rubbers, e.g. Neoprene® from DuPont; nitrile butadiene rubbers, e.g. Buna N®; polybutadiene; polytetrafluoroethylene; modified and unmodified celluloses, homopolymerisates and copolymerisates of alpha-olefins and copolymers composed of two or more monomer units forming the aforementioned polymers; polyvinyl alcohols, polyalkylene oxides, e.g. polyethylene oxides; poly-N-vinylpyrrolidone; hydroxymethyl celluloses; maleic acids; alginates; collagens.

All the aforementioned polymers may be used individually or in any combination with one another in the electrospun polymer fibers according to the present invention, namely in any mixing ratio.

In a preferred embodiment, the electrospun polymer fiber comprises poly-L-lactide (PLLA), polystyrene (PS) or polyvinyl butyral (PVB).

The electrospun polymer fibers comprising particles of bacteria-containing hydrogels are produced by a method comprising the following steps:
producing a solution of bacteria-containing hydrogel particles and at least one electrospinnable polymer in an organic solvent or a mixture of organic solvents,
electrospinning this solution,
   wherein the hydrogel particles are physically or chemically crosslinked.

As mentioned in the introduction, a hydrogel is a water-containing yet water-insoluble polymer, the molecules of which are chemically or physically linked to form a three-dimensional network.

The hydrogel particles containing bacteria may be physically or chemically crosslinked.

Particles of bacteria-containing, physically crosslinked hydrogels are produced according to the present invention by a method comprising the following steps:
a) producing an aqueous solution of a water-soluble polymer,
b) producing a sediment of an aqueous liquid culture of the bacteria,
c) bringing the polymer into contact with the sediment of the liquid culture of the bacteria,
d) stirring the mixture from step d) at high speed,
e) physically crosslinking the polymer,
f) using filtration to remove the bacteria-containing hydrogel particles that are formed.

The physical crosslinking according to step e) advantageously occurs by repeated thawing and freezing as described above.

Step e) of the above method may optionally be carried out before step c), so that the physical crosslinking takes place before the bacteria are added.

In a preferred embodiment, the aqueous solution of a water-soluble polymer is an aqueous solution of polyvinyl alcohol.

Particles of bacteria-containing, chemically crosslinked hydrogels are produced according to the present invention by a method comprising the following steps:
a) producing a solution of a water-soluble polymer,
b) chemically crosslinking the water-soluble polymer to form the hydrogel and swelling this hydrogel,
c) producing a sediment of an aqueous liquid culture of the bacteria,
d) bringing the polymer into contact with the sediment of the liquid culture of the bacteria,
e) stirring the mixture from step d) at high speed,
f) using filtration to remove the bacteria-containing hydrogel particles that are formed.

It is known to persons skilled in the art how a chemical crosslinking according to step b) of the above method is to be carried out. Suitable crosslinkers have already been mentioned.

In a further embodiment, chemically crosslinked, bacteria-containing hydrogel particles are produced by carrying out step e) according to the above method before step d). Therefore, in this embodiment particles of the chemically crosslinked hydrogel are produced first of all and then the bacteria are incorporated.

Furthermore, it is known to persons skilled in the art how liquid cultures of bacteria and also sediments of these liquid cultures have to be produced. Persons skilled in the art are able to apply this knowledge without leaving the scope of protection of the patent claims.

If hydrogel particles of physically crosslinked polyvinyl alcohol are to be produced, the solution of the polyvinyl alcohol according to step a) of the aforementioned method for producing physically crosslinked hydrogel particles advantageously has a concentration of 10 wt.-% to 20 wt.-%.

The solution is advantageously incorporated in a phase which stabilizes the particle precursors. This phase may be, for example, a silicone oil, such as a phenylmethyl silicone such as AP200®.

The aqueous solution of the polyvinyl alcohol and the sediment of the liquid culture of the bacteria are advantageously mixed with one another in a ratio of 6:1 (w/w).

Both physically and chemically crosslinked hydrogel particles are produced by high-speed stirrers, this advantageously being understood to mean stirring speeds of 5000 to 15,000 rpm.

The bacteria-containing hydrogel particles that are formed are then removed by filtration.

Electrospinning is known per se. In this process, a solution of the polymer that is to be spun is exposed to a high electric field at an edge serving as electrode. By way of example, this may take place, in an electric field and at low pressure, by extruding the solution that is to be spun through a cannula connected to a pole of a voltage source. A material flow directed toward the counter-electrode is obtained, which solidifies on the way to the counter-electrode.

The spinning solution may optionally comprise further components in addition to the polymer or polymer mixture. In the case of the present invention, the spinning solution additionally comprises the hydrogel particles containing bacteria.

During the spinning process, a frame made from a conductive material, for example a rectangular frame, may be introduced between the nozzle and the counter-electrode. In this case, the fibers are deposited on this frame in the form of an oriented nonwoven. This method of producing oriented mesofiber and nanofiber nonwovens is known to persons skilled in the art and is suitable to be used without departing from the scope of protection of the claims.

According to step h) of the method according to the present invention, a solution of at least one electrospinnable polymer and the hydrogel particles from step g) is electrospun.

The spinning solution is advantageously produced by firstly predispersing the hydrogel particles from step g) in an organic solvent or in a mixture of organic solvents and then adding a solution of the electrospinnable polymer. It is advantageous to dissolve the electrospinnable polymer in the same solvent or solvent mixture as the hydrogel particles.

It is known to persons skilled in the art which organic solvents are suitable for the electrospinning process. Suitable solvents are for example dichloromethane, ethanol, chloroform and mixtures of these solvents.

In the electrospun polymer fibers according to the present invention comprising particles of bacteria-containing hydrogels, bacteria are suitable to be stored alive in the dry state for a long period of time (for at least one year). Here, “dry” means that water or a cell culture medium need not be present in, or added to, the electrospun polymer fibers comprising particles of bacteria-containing hydrogels in order to keep the bacteria alive. When required, the bacteria stored in this way are suitable to be reactivated by wetting with water or a cell culture medium, this reactivation being recognizable by the bacteria starting to multiply.

On the other hand, in the electrospun polymer fibers according to the present invention comprising bacteria-containing hydrogels, bacteria are protected against solvents which would otherwise kill said bacteria. These solvents comprise for example ethanol, propanol, acetone, dichloromethane, chloroform, toluene and tetrahydrofuran (THF). Bacteria which are “packaged” in the device according to the present invention can even be processed from these solvents.

It should be emphasized that the “packaging” of bacteria in hydrogels, as described in the context of the present invention, leads to the situation in which bacteria packaged in this way are suitable to be stored in the dry state, are protected against said solvents which would otherwise kill them, and are suitable to be processed from said solvents. However, if these bacteria-containing hydrogel particles are additionally incorporated in electrospun polymer fibers, they can be better handled in the applications mentioned here.

In one embodiment of the present invention, the electrospun polymer fibers according to the present invention comprising bacteria-containing hydrogels are therefore used for storing bacteria in the dry state. This storage is advantageous since it is thus possible to save on the considerable costs of the otherwise customary storage methods, for example for the procurement and maintenance of cell culture cabinets and culture vessels, of freeze-drying apparatuses, the freezing and storage of bacteria at very low temperatures and the regular supply of cell culture media to the bacteria.

In one specific embodiment, electrospun polymer fibers according to the present invention comprising bacteria-containing hydrogels are suitable for use in textile finishes and for incorporation in membranes. The bacteria which are “stored” in this way in the textiles or membranes are preferably useful bacteria which carry out desired metabolic processes or which kill pathogenic germs.

By way of example, a layer of electrospun polymer fibers according to the present invention comprising bacteria-containing hydrogels can be stored in the membrane between two electrospun nonwovens without hydrogel particles. Such membranes may optionally be applied to a support material, for example a plastic or paper filter, and then used to remove by filtration pathogenic bacteria from aqueous media. Upon contact with water, the useful bacteria are released from the hydrogels and kill the pathogenic bacteria retained in the filter membrane.

In a further embodiment, the electrospun polymer fibers according to the present invention comprising bacteria-containing hydrogels are suitable for use in equipping cosmetic products. For example, hygiene products such as diapers and incontinence pads can be equipped in this way with useful bacteria which kill pathogenic germs and/or odor-causing bacteria.

In a further embodiment, the electrospun polymer fibers according to the present invention comprising bacteria-containing hydrogels are suitable for use in bacterial fuel cells.

In general, there are a wide range of possible uses for the electrospun polymer fibers according to the present invention comprising bacteria-containing hydrogels. These areas of application may differ for allowing useful bacteria to live as functional units on the one hand and for killing harmful bacteria on the other hand. This device according to the present invention is thus suitable for use for example in the aforementioned applications in textiles and membranes, as well as in applications in wastewater treatment, environmental protection (water pollution control), the agricultural and food sector, pharmacy, fermentation, and the building industry.

### LIST OF REFERENCE NUMERALS

1 voltage source

2 capillary nozzle

3 syringe

4 spinning solution

5 counter electrode

6 fiber formation

7 fiber mat

### FIGURE LEGENDS

FIG. 1

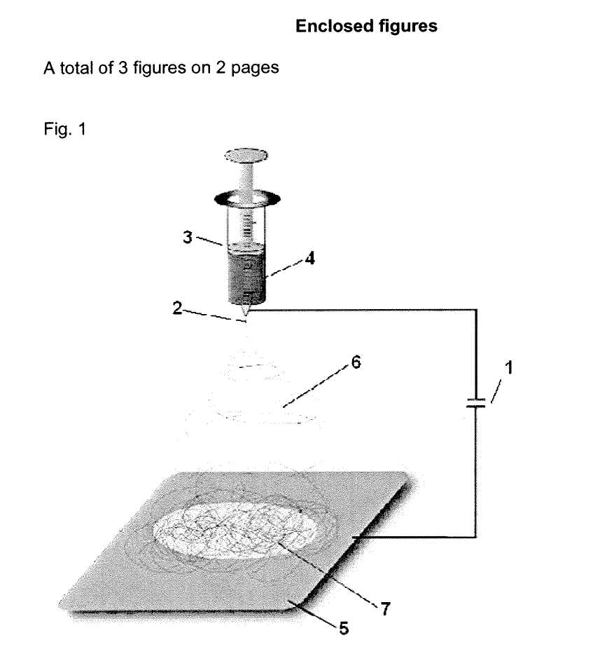

FIG. 1 shows a schematic representation of a device suitable for carrying out the electrospinning process.

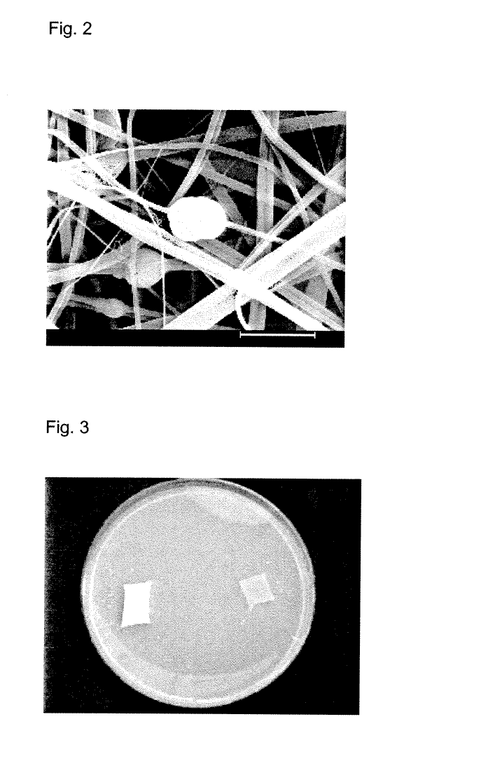

The device comprises a syringe 3, at the tip of which a capillary nozzle 2 is located. This capillary die 2 is connected to a pole of a voltage source 1. The syringe 3 takes up the solution 4 to be spun. Arranged at a distance of approximately 20 cm opposite the outlet of the capillary nozzle 2 is a counter-electrode 5 which is connected to the other pole of the voltage source 1 and which acts as a collector for the fibers that are formed.

During operation of the device, a voltage of between 18 kV and 25 kV is set on the electrodes 2 and 5 and the spinning solution 4 is discharged through the capillary nozzle 2 of the syringe 3 under low pressure. Due to the electrostatic charge of the polymer molecules in the solution resulting from the strong electric field of 0.9 to 2 kV/cm, a material flow directed toward the counter-electrode 5 occurs, and which solidifies on the way to the counter-electrode 5, resulting in fiber formation 6, as a result of which fibers 7 having diameters in the micrometer and nanometer scale are deposited on the counter-electrode 5.

FIG. 2

The figure shows M. luteus-containing particles of physically crosslinked polyvinyl alcohol embedded in PVB fibers. The white bar at the bottom edge of the figure corresponds to a length of 3.00 μm.

FIG. 3

The figure shows the bacterial lawn after incubating a fiber mat (as described in FIG. 3) on an agar plate.

### PRACTICAL EMBODIMENTS

### Practical Embodiment 1

### Producing Hydrogel Particles

In order to produce the hydrogel particles, a mixture of one milliliter of a solution of 10% by weight of polyvinyl alcohol 56-98 (KSE) in water was dispersed in 80 g of silicone oil (AP200, Wacker). For this, use was made of a high-speed stirrer IKA® T18 basic Ultra-Turrax® with a dispersing tool S 18N-19G at 10,000 rpm. The treatment time was ten minutes. The resulting dispersion was then frozen at −20° C. After 20 hours at −20° C., the dispersion was stored for four hours at room temperature. This cycle was repeated twice. After the final thawing, the dispersion was added to three times the quantity of acetone, with rapid stirring. The hydrogel particles, which had now collapsed, could then be removed by filtration.

### Practical Embodiment 2

### Producing Bacteria Immobilized in Hydrogel Particles

The bacteria immobilized in the hydrogel particles were Escherichia (E.) coli and Micrococcus (M.) luteus. E. coli was cultured in a nutrient solution of 30 g of tryptic soy broth in 1000 ml of water, and M. luteus was cultured in a mixture of 5.0 g of meat extract and 3.0 g of peptone in 1000 ml of water at pH=7. The bacteria were sedimented and washed with 50 mmol/l phosphate buffer pH=7.

In order to produce bacteria-containing hydrogel particles, the bacteria were added in the form of the sediment of a liquid culture to the polyvinyl alcohol solution immediately prior to processing. Here, 0.5 g of sediment was used for 3 g of PVA solution.

### Practical Embodiment 3

### Detecting Living Bacteria in the Hydrogel Particles

The detection of living bacteria in the hydrogel particles took place by applying such particles to agar plates. In each case the agar plates comprised the nutrient media described above, to which agar-agar had been added for solidification purposes. The agar plates were then incubated at 37° C. for at most 72 h, whereby bacterial growth could be seen in the region of the applied particles. Samples of the growth were removed from the plates, cultured again on fresh agar plates and subjected to microscopic analysis. It was able to be confirmed that the bacteria in question were the previously immobilized E. coil and M. luteus. The particles were stored at 4° C. in closed vessels in the absence of light. At various points in time, particles were again applied to agar plates in order to monitor in qualitative terms the ability of the bacteria to survive in the particles for a relatively long period of time.

The results are shown in Table 1.

TABLE 1
Ability to survive when stored at 4° C.

| Storage/months | M. luteus | E. coli |
|---|---|---|
| 0 | alive | alive |
| 1 | alive | alive |
| 2 | alive | alive |
| 3 | alive | alive |
| 4 | alive | alive |
| 5 | alive | alive |
| 6 | alive | alive |
| 7 | alive | alive |
| 8 | alive | alive |
| 9 | alive | alive |
| 10 | alive | alive |

### Practical Embodiment 4

### Protecting the Bacteria in Hydrogel Particles Against Organic Solvents

The particles obtained were tested with regard to their property of protecting the bacteria contained therein against organic solvents. For that purpose, samples of the particles were stored in small volumes of said solvents. In order to detect living bacteria in these particles, samples were taken using a pipette. These samples were then left to dry on a sterile slide in order to remove the solvent. The slide was then placed on an agar plate and removed again once the particles had swelled, with the particles remaining on the agar surface. Growth in the region of the slide indicated the presence of living bacteria from the particles. The solvents tested were acetone, ethanol, chloroform, dichloromethane, tetrahydrofurane and toluene. A mixture of acetone with 15% water was also tested.

The results are shown in Table 2.

TABLE 2
Ability to survive in various solvents

| Solvent | Residence time/h | E. coli | M. luteus |
|---|---|---|---|
| Dichloromethane | 0.5 | alive | alive |
| | 24 | alive | alive |
| | 144 | alive | alive |
| | 264 | alive | alive |
| Ethanol | 0.5 | alive | alive |
| | 24 | alive | alive |
| | 144 | alive | alive |
| | 264 | alive | alive |
| Tetrahydrofurane | 0.5 | alive | alive |
| | 24 | alive | alive |
| | 144 | alive | alive |
| Chloroform | 0.5 | alive | alive |
| | 24 | alive | alive |
| | 144 | alive | alive |
| | 264 | alive | alive |
| Toluene | 0.5 | alive | alive |
| | 24 | alive | alive |
| | 144 | alive | alive |
| | 264 | alive | alive |
| 15% water in acetone | 1 | dead | dead |

### Practical Embodiment 5

### Incorporating the Bacteria-Containing Hydrogel Particles in Polymer Fibers

The incorporation of the particles in polymer fibers took place by means of the electrospinning technique. The entire apparatus was sterilized as far as possible by wiping it with 70 vol % ethanol in order to reduce the likelihood of contamination of the samples. To date, bacteria-containing particles have been spun in poly-(L-lactide) (PLLA) and polyvinyl butyral (PVB) and polystyrene (PS).

In the case of PLLA, dichloromethane was used as solvent. The particles were first predispersed in a small quantity of solvent with the aid of ultrasound. A more concentrated solution of PLLA in dichloromethane was then added, so that the total concentration was 4 wt.-% PLLA. The concentration of the particles in the solution was approx. 10 mg per gram of solution. The solution was spun using an electrode gap of 20 cm and a voltage of 25 kV. The flow rate was 0.9 ml/h. The procedure was the same in the case of polyvinyl butyral and polystyrene. Ethanol and chloroform were used as solvents.

The spinning conditions are shown in Table 3.

TABLE 3
Conditions for spinning the particles with different polymers.

| | [c]/ | Voltage/ | Gap/ | Flow/ |
|---|---|---|---|---|
| Polymer | % by weight | kV | cm | ml/h |
| PLLA | 4 | 25 | 20 | 0.9 |
| PVB | 11 | 25 | 20 | 0.9 |
| PS | 13 | 25 | 20 | 0.5 |

1. Electrospun polymer fibers comprising particles of bacteria-containing hydrogels.

2. Electrospun polymer fibers according to claim 1, wherein the hydrogels comprise polyvinyl alcohol, polyethylene oxide, polyethyleneimine, polyvinylpyrrolidone, polyacrylic acid, methyl cellulose, hydroxypropyl cellulose, polyacrylamide, starch or partially saponified cellulose acetate, in each case in crosslinked form.

3. Electrospun polymer fibers according to claim 1, wherein the hydrogels comprise crosslinked polyvinyl alcohol.

4. Electrospun fibers according to claim 1, wherein the electrospun polymer fiber comprises poly-L-lactide (PLLA), polystyrene (PS) or polyvinyl butyral (PVB).

5. A method for producing electrospun polymer fibers comprising bacteria-containing hydrogels according to claim 1, comprising the steps:
a) producing a solution of bacteria-containing hydrogel particles and at least one electrospinnable polymer in an organic solvent or a mixture of organic solvents, and
b) electrospinning this solution, wherein the hydrogel particles are physically or chemically crosslinked.

6. The method according to claim 5, wherein the hydrogel particles are physically crosslinked and produced through a method comprising the steps:
producing an aqueous solution of a water-soluble polymer,
producing a sediment of an aqueous liquid culture of the bacteria,
bringing the polymer into contact with the sediment of the liquid culture of the bacteria,
stirring the mixture from step d) at high speed,
physically crosslinking the polymer, and
using filtration to remove the bacteria-containing hydrogel particles that are formed.

7. The method according to claim 6, wherein the aqueous solution of the water-soluble polymer is an aqueous solution of polyvinyl alcohol.

8. Use of electrospun polymer fibers comprising bacteria-containing hydrogels according to claim 1 for the storage of bacteria in the dry state.

9. Electrospun polymer fibers according to claim 3, wherein the hydrogels comprise physically crosslinked polyvinyl alcohol.
